SEQUENCE LISTING (1 sequence)

SEQ ID NO: 1 - DNA, 2606 bp, synthetic construct
  source 1..2606: mol_type = other DNA
gacggctgtcactcagcctgacggacagcccagagatcggccttcagataaaaccttacagatgtggaggtcggtattgattttgagttagtaacggtaaggtaccaagaagtaatacattagtgaaagcaaggtcactgggaaaaaggtaaaacccactgggggagaggccagggcattcttgaggggggtgatgaccgcatcagatcaggacttcctcaagttcaaaagtttgcaggccccagacctctggtgggcccctaactgtgggccaacgcttcctgcttccctgggcgaagggggacccgtgggggtctgtcagccgcccgagactggcgagggccgtgtaggcctgtggagctctgccctgcggggctgccgtgggcctctctccccacgctctctggctcagcttgctgtggggagggatcacagcgaggggtgaggagacacgggttcaagtcccagtctgccacgacctggcggtgcagtctcaggcactccttttcacgtccctgtgccttgatttccccatctgtagaatgggggtaaaacagctcatttgccggagaagtcattccacgctccacctctctttagaggggtctgaccccatcagtttggaagcgctcatgcatgccttgagacaggaggctggacgcaggcaaaggcctctgatccccagacttccgggcaccctggaagagagcgagcagatgctgcacaccccccattcacacccagcccagatgtggacctgatttgatgttgcagggcaaatcccactgaacaaatcccagtacagggaaagctccccgggcctgagcagattctgagctgtctgttaatggtgcagggtccaagcccccccaagccactgggcaccacacgaaccccttctcacagagcattcccggaaggttagcatcgaccttcttgctgtgtctcagatctgctaggcgtgttccagcctttgcacttgctggctcttctgcctggacaactctctgttctgtcttcccctggctgattctttctggtcttgctagtgaggtcttccctgactactccacatattttttcttccatagcacttatcagtattggaaattatattatctgttttcatcttcctacagttgactgactggcagctcctgaaggtcatggagctgttagtcttcattcctgaatttcaatgcctggtacagtgtctggcacatattaggtgctcaataaatatttgttaagtgaacatgcatgtatttgacagggttttctgagcttatagtttgcagggggtacagtgtagccaccggggctgcagtgatggatgaaacacaatttcattctgccctcagagagctcacagctgagccctgtctcccgcctggcatataataacgctctgtgaagtgtcttatgaatgaaggaaggatgtctttaggaaacactatgtggtttgggacagctgggacagaaagtgcaaggtaggaaggaggcagggatttgggacaggcaagtggaggtccagtcccacgaagcttcgccctgttcatctgggaggttccaaagagccacgggtaattcttgagcaacgtggtcagatttgttgggcggctgttgccttgcaggatgagtatggatctatgcacaccttccagcctgtcctccaaagcatccttctcctctccccaaatacctccccacttgtctcctccatcccatccttgctgggcctggcgtcttggtgatatgtaaagagaaggattataccagaggctcatcagaactgacaaaccctcatgagaggcttcccaccccacaacgatcctttcaaagaaagagaaatgggacctgtgtgttttccctgagcctctaagtttgttcttggggccagggctccttgaaggaggagcagtgttagcaatgaatcaggtctcctaaccccagggcagtgtcagcctcctgagtgcccccggccccgcagacccaccaaacacacacacccagcaaactcctcaggcccaggaccccaccgcaacagtcaggaaagaacaaacccagctccacctgtgtttctgccatctcattgggctggcaccctgcaaagtcccacccaaacctcaccagctgcggtggagacaaagtccagccccagtaggcctggtgcccttgacttcagctctgggggtggggtgaggaaaggacagaggggttgagagaaaggctgagaggtgcacgctgggtgcattgcaggaagagtcaggggaggctgtgctcgtggtattcttgaggagccaggccccctgcgggcctgggatcccaccccacacccccagggaacccccatctggagtgagaacctctatcatggtcacgtgtctaaaacccatccttgagagtcagacttgacatcatcaggctagaggggtcaagccctggcacacccgctgtgagatctggggaagacgcacctctgggtgggcctcggtctcttcatctgagaaatgggcgtcatgactccgccccacacgtctctggagggctctgcactggcagagtcacgccgagtctgttgctaagaggagggaaagggaaagagg

(19) United States
(12) Patent Application Publication
(10) Pub. No.: US 2024/0360440 A1
(43) Pub. Date: Oct. 31, 2024

(54) Bos GRUNNIENSLNC4047 AND APPLICATION THEREOF

(71) Applicant: LANZHOU INSTITUTE OF HUSBANDRY AND PHARMACEUTICAL SCIENCES OF CAAS., Lanzhou City (CN)

(72) Inventors: Xiaoming Ma, Lanzhou City (CN); Ping Yan, Lanzhou City (CN); Chunnian Liang, Lanzhou City (CN); Xian Guo, Lanzhou City (CN); Tong Wang, Lanzhou City (CN)

(21) Appl. No.: 18/140,060

(22) Filed: Apr. 27, 2023

(51) Int. Cl. C12N 15/11; C12N 5/077

(52) U.S. Cl. CPC C12N 15/11; C12N 5/0658

(57) ABSTRACT

The present invention provides a bos grunniens Lnc4047 and applications thereof, and belongs to the technical field of genetic engineering. The nucleotide sequence of the bos grunniens Lnc4047 is shown in SEQ ID No. 1. The regulation effect of Lnc4047 on myoblast proliferation and myoblast differentiation of bos grunniens is discovered in the present invention for the first time, that is, Lnc4047 inhibits myoblast proliferation, but promotes myoblast differentiation to a certain extent. This provides an important basis for understanding the muscle development mechanism of bos grunniens, and lays a theoretical foundation for rapidly developing a new high-quality breed of bos grunniens and improving its meat quality.

### TECHNICAL FIELD

The present invention relates to the technical field of genetic engineering, in particular to a bos grunniens Lnc4047 and applications thereof.

### BACKGROUND

Bos grunniens is a herbivorous ruminant domestic animal in bovinae, bovidae, mammalia. This species of animal reside in high altitude areas with cold and hypoxia conditions ranging from 3 km to 6 km all year round. Bos grunniens is a mammal which is primarily distributed in plateau areas subjected to an extremely challenging natural environment, featuring low annual mean temperatures (lower than or equal to 0° C.), significant temperature fluctuations between day and night (above 15° C.), short growing seasons for pastures (110-135d), strong radiation (above 140-195 KJ/cm2 annularly), low oxygen pressure (below 110 mm Hg), etc. Due to the special ecological environment and strong natural selection, bos grunniens has tenacious vitality, and has a unique physical structure and physiological mechanism for resistance to roughages and cold. This not only sets bos grunniens apart from other cattle breeds in terms of body performance and physique, but also affects the yield, quality, and nutritional content of meat products derived from bos grunniens. In 2019, around 3.6 million bos grunnienses were slaughtered nationwide, with an average carcass weight of about 128 kg per head. This yielded approximately 470,000 tons of carcass weight with a net meat output of 370,000 tons, valued at approximately 27 billion yuan. In recent years, bos grunniens has become a beloved green food choice for the public due to their delicious meat, rich nutritional content, various amino acids, and high protein and low fat. However, their unique living environment and 7-month hay period lead to fluctuations in their growth and weight gain, which negatively affects their economic and utilitarian values. The growth rate and meat production of domestic animals are closely associated with the growth and development of skeletal muscles. The rapid development of the second-generation sequencing technology allows for high-throughput, quick and low-cost sequencing of hundreds of thousands to millions of DNA molecules at one time, making genome or transcriptome sequencing more accessible. Transcriptome sequencing is fundamental for gene function and structure research, and can solve various problems, such as exploring new genes, discovering low-abundance transcripts, mapping transcription, identifying gene families, and analyzing evolution. It has applications in various fields, such as biology, medicine, and agronomy. Therefore, the use of new technological approaches to identify the major genes affecting the growth traits of bos grunniens is particularly important for the healthy development of bos grunniens industry.

The production traits of bos grunniens are primarily manifested in the growth and development of muscles. The growth speed and quantity of muscles, especially the growth traits of skeletal muscles, directly affect the meat yield of bos grunniens. Skeletal muscle development is a complex physiological process that involves a series of developmental events resulting from the interaction between environmental factors and genetic factors. This process includes various transcription regulatory factors, signal pathways, and complex regulatory mechanisms, which have a critical impact on animal growth and development. The proliferation and differentiation of myoblasts has been a research hotspot in recent years. Skeletal muscles are not only an integral part of animal growth and development but also play a crucial role as a carrier of animal energy metabolism. Studies have found that lncRNAs are involved in skeletal muscle development and fiber-type transformation. However, previous studies primarily focused on humans and mice, with few reports on the functions and mechanisms of lncRNAs related to skeletal muscle development of domestic animals. There are numerous lncRNAs, and their action mechanisms are diverse, with regulatory mechanisms in different species varying widely. Therefore, accurately determining the mechanism of action of lncRNAs in different species is essential.

### SUMMARY

In order to solve the problem described above, a bos grunniens Lnc4047 and applications thereof are provided in the present invention. The regulation effect of Lnc4047 on myoblast proliferation and myoblast differentiation of bos grunniens is discovered in the present invention for the first time, that is, Lnc4047 inhibits myoblast proliferation, but promotes myoblast differentiation to a certain extent. This provides an important basis for understanding the muscle development mechanism of bos grunniens, and lays a theoretical foundation for rapidly developing a new high-quality breed of bos grunniens and improving their meat quality.

To achieve the above objective, the present invention provides the following technical solution.

A bos grunniensLnc4047 is provided in the present invention, and the nucleotide sequence of the bos grunniensLnc4047 is shown in SEQ ID No. 1.

Further provided in the present invention is an application of the bos grunniens Lnc4047 described in the above technical solution in regulating myoblast proliferation of bos grunniens.

Preferably, an overexpressed bos grunniens Lnc4047 inhibits myoblast proliferation of bos grunniens.

Preferably, a silent bos grunniens Lnc4047 promotes myoblast proliferation of bos grunniens.

Further provided in the present invention is an application of the bos grunniens Lnc4047 described in the above technical solution in regulating myoblast differentiation of bos grunniens.

Preferably, the overexpressed bos grunniens Lnc4047 promotes myoblast differentiation of bos grunniens.

Preferably, the silent bos Lnc4047 promotes myoblast proliferation of bos grunniens.

The beneficial effects are as follows:

The regulation effect of Lnc4047 on myoblast proliferation and myoblast differentiation of bos grunniens is discovered in the present invention for the first time, that is, Lnc4047 inhibits myoblast proliferation, but promotes myoblast differentiation to a certain extent. This provides an important basis for understanding the muscle development mechanism of bos grunniens, and lays a theoretical foundation for rapidly developing a new high-quality breed of bos grunniens and improving its meat quality.

### BRIEF DESCRIPTION OF DRAWINGS

The drawings required for use in the example will be briefly described hereinafter in order to more clearly explain the example of the present invention or the technical solutions in the prior art.

FIG. 1 shows the purification of primary skeletal muscle myoblasts of bos grunniens using the Percoll method.

FIG. 2 shows the expression of Lnc4047 in cell proliferation and differentiation stages, and the expression distribution of nuclear extraction in different stages.

FIG. 3 shows the effects of Lnc4047 on the myoblast proliferation of bos grunniens measured using CCK8, Edu and a flow cytometer.

FIG. 4 shows the expressions and protein abundance changes of myodifferentiation marker genes MyF5 and MyoG after Lnc4047 interference/overexpression.

FIG. 5 shows the in-situ expression levels of MyoG and MyF5 measured using immunofluorescence after Lnc4047 interference/overexpression.

### DETAILED DESCRIPTION

A bos grunniens Lnc4047 is provided in the present invention, and the nucleotide sequence of the bos grunniensLLnc4047 is shown in SEQ ID No. 1, specifically as follows:

GACGGCTGTCACTCAGCCTGACGGACAGCCCAGAGATCGGCCTTCAGATA
AAACCTTACAGATGTGGAGGTCGGTATTGATTTTGAGTTAGTAACGGTAA
GGTACCAAGAAGTAATACATTAGTGAAAGCAAGGTCACTGGGAAAAAGGT
AAAACCCACTGGGGGAGAGGCCAGGGCATTCTTGAGGGGGGTGATGACCG
CATCAGATCAGGACTTCCTCAAGTTCAAAAGTTTGCAGGCCCCAGACCTC
TGGTGGGCCCCTAACTGTGGGCCAACGCTTCCTGCTTCCCTGGGCGAAGG
GGGACCCGTGGGGGTCTGTCAGCCGCCCGAGACTGGCGAGGGCCGTGTAG
GCCTGTGGAGCTCTGCCCTGCGGGGCTGCCGTGGGCCTCTCTCCCCACGC
TCTCTGGCTCAGCTTGCTGTGGGGAGGGATCACAGCGAGGGGtgaggaga
cacgggttcaagtcccagtctgccACGACCTGGCGGTGCAGTCTCAGGCA
CTCCTTTTCACGTCCCTGTGCCTtgatttccccatctgtagaatgggggt
aAAACAGCTCATTTGCCGGAGAAGTCATTCCACGCTCCACCTCTCTTTAG
AGGGGTCTGACCCCATCAGTTTGGAAGCGCTCATGCATGCCTTGAGACAG
GAGGCTGGACGCAGGCAAAGGCCTCTGATCCCCAGACTTCCGGGCACCCT
GGAAGAGAGCGAGCAGATGCTGCACACCCCCCATTCACACCCAGCCCAGA
TGTGGACCTGATTTGATGTTGCAGGGCAAATCCCACTGAACAAATCCCAG
TACAGGGAAAGCTCCCCGGGCCTGAGCAGATTCTGAGCTGTCTGTTAATG
GTGCAGGGTCCAAGCCCCCCCAAGCCACTGGGCACCACACGAACCCCTTC
TCACAGAGCATTCCCGGAAGGTTAGCATcgaccttcttgctgtgtctcaG
ATCTGCTAGGCGTGTTCcagcctttgcacttgctggcTCTTCTGCCTGGA
CAACTCTCTGTTCTGTCTTCCCCTGGCTGATTCTTTCTGGTCTTGCTAGT
GAGGTCTTCCCTGACTACTCcacatattttttcttccatagCACTTATCA
GTATTGGAAATTATATTATCTGTTTTCATCTTCCTACAGTTGACTGACTG
GCAGCTCCTGAAGGTCATGGAGCTGTTAGTCTTCATTCCTGAATTTCAAT
GCCTggtacagtgtctggcacatattaggtgctcaataaatatttgttaa
gtgaacATGCATGTATTTGACAGGGTTTTCTGAGCTTATAGTTTGCAGGG
GGTACAGTGTAGCCACCGGGGCTGCAGTGATGGATGAAACACAATTTCAT
TCTGCCCTCAGAGAGCTCACAGCTGAGCCCTGTCTCCCGCCTGGCATATA
ATAACGCTCTGTGAAGTGTCTTATGAATGAAGGAAGGATGTCTTTAGGAA
ACACTATGTGGTTTGGGACAGCTGGGACAGAAAGTGCAAGgtaggaagga
ggcagggatTTGGGACAGGCAAGTGGAGGTCCAGTCCCACGAAGCTTCGC
CCTGTTCATCTGGGAGGTTCCAAAGAGCCACGGGTAATTCTTGAGCAACG
TGGTCAGATTTGTTGGGCGGCTGTTGCCTTGCAGGATGAGTATGGATCTA
TGCACACCTTCCAGCCTGTCCTCCAAAGcatccttctcctctccccaaaT
ACCTCCCCACTTGTCTCCTCCATCCCATCCTTGCTGGGCCTGGCGTCTTG
GTGATATGTAAAGAGAAGGATTATACCAGAGGCTCATCAGAACTGACAAA
CCCTCATGAGAGGCTTCCCACCCCACAACGATCctttcaaagaaagagaa
atgggacCTGTGTGTTTTCCCTGAGCCTCTAAGTTTGTTCTTGGGGCCAG
GGCTCCTTGAAGGAGGAGCAGTGTTAGCAATGAATCAGGTCTCCTAACCC
CAGGGCAGTGTCAGCCTCCTGAGTGCCCCCGGCCCCGCAGacccaccaaa
cacacacacccagcaaACTCCTCAGGCCCAGGACCCCACCGCAACAGTCA
GGAAAGAACAAACCCAGCTCCACCTGTGTTTCTGCCATCTCATTGGGCTG
GCACCCTGCAAAGTCCCACCCAAACCTCACCAGCTGCGGTGGAGACAAAG
TCCAGCCCCAGTAGGCCTGGTGCCCTTGACTTcagctctgggggtggggt
gaggaaagGACAGAGGGGTTGAGAGAAAGGCTGAGAGGTGCACGCTGGGT
GCATTGCAGGAAGAGTCAGGGGAGGCTGTGCTCGTGGTATTCTTGAGGAG
CCAGGCCCCCTGCGGGCCTGGGatcccaccccacacccccaggGAACCCC
CATCTGGAGTGAGAACCTCTATCATGGTCACGTGTCTAAAACCCATCCTT
GAGAGTCAGACTTGACATCATCAGGCTAGAGGGGTCAAGCCCTGGCACAC
CCGCTGTGAGATCTGGGGAAGACGCACCTCTGGGTGGGCCTCGGTCtctt
catctgagaaatgggcGTCATGACTCCGCCCCACACGTCTCTGGAGGGCT
CTGCACTGGCAGAGTCACGCCGAGTCTGTTGCTAAGAggagggaaaggga
aagagg.

Further provided in the present invention is an application of the bos grunniens Lnc4047 described in the above technical solution in regulating myoblast proliferation of bos grunniens. In the present invention, an overexpressed bos grunniens Lnc4047 preferably inhibits myoblast proliferation of bos grunniens. In the present invention, a silent bos grunniens Lnc4047 preferably promotes myoblast proliferation of bos grunniens.

Further provided in the present invention is an application of the bos grunniens Lnc4047 described in the above technical solution in regulating myoblast differentiation of bos grunniens. In the present invention, an overexpressed bos grunniens Lnc4047 preferably promotes myoblast differentiation of bos grunniens. In the present invention, a silent bos grunniens Lnc4047 preferably promotes myoblast proliferation of bos grunniens.

To further illustrate the present invention, the present invention will be hereinafter described in detail in conjunction with an example, which, however, should not be understood as a limitation to the scope of protection of the present invention.

### Example 1

### 1. Separation Method:

A newborn calf was killed according to standard procedures. The hair and skin of a sampling area were removed to expose skeletal muscle tissue. The tissue sample was transferred into 1×PBS containing 10% of a bispecific antibody with a sterile surgical instrument. The sample was quickly transferred into a sterile cell chamber, and the connective tissue and blood vessels in the muscle were removed with a sterile surgical instrument. The sample was placed in a proper amount of 1×PBS, and the tissue was cut into pieces with a sterile surgical instrument. An appropriate amount of a collagenase Type IV solution was added into the cut tissue sample, and the tissue was further cut in the presence of digestive enzymes. After the above two cutting processes, the muscle tissue was basically turned into mince. Then, the skeletal muscle tissue mince was transferred into a centrifuge tube and digested in a 37° C. water bath for 20 min. During this period, the tissue was gently blown multiple times with a pipette to ensure that digestion was more thorough.

### 2. Purification Mode:

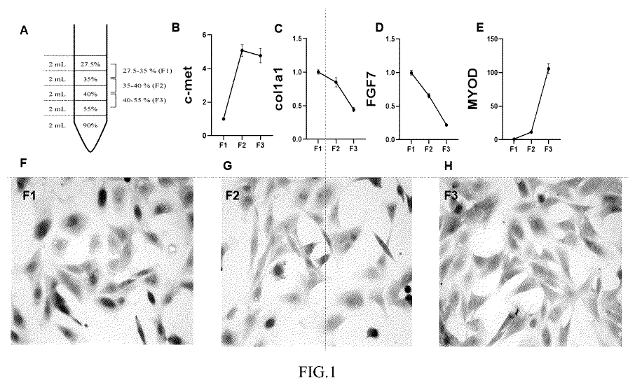

Since the separated skeletal muscle cells mainly contained myoblasts and fibroblasts, a five-step Percoll gradient (A in FIG. 1) was used in the present invention. The collected cells with Percoll fractions of 27.5-35%, 35-40% and 40-55% were defined as F1, F2 and F3, respectively. The expression levels of two myoblast marker genes (MyoD and c-met) and two fibroblast marker genes (FGF7 and collal) were analyzed using qPCR. The results showed that compared with F1 and F2, F3 had a higher myoblast marking level and a lower fibroblast marking level (B-E in FIG. 1), indicating the existence of purified myoblasts in F3. In addition, the differentiation states of these cell components were also examined. F3 had the most abundant myotubes compared with the other components (F-H in FIG. 1), indicating the existence of myogenic cells in F3. Therefore, it was proved that myoblasts separated from the longest dorsal muscle of bos grunniens could be purified using Percoll density gradient centrifugation without any expensive equipment or special technique.

### 3. Cell Passage and Cryopreservation

### Terminating Culture Medium DMEM+10% FBS (Fetal Bovine Serum)

| | |
|---|---|
| DMEM (sigma D6429) | 45 mL |
| FBS | 5 mL |
| Total | 50 mL |

### Myoblast Culture Medium DMEM/F-12+20% FBS+1% Bispecific Antibody (Penicillin-Streptomycin)

| | | |
|---|---|---|
| DMEM/F-12 (gibco 11330032) | 39.5 | mL |
| FBS | 10 | mL |
| Bispecific antibody | 500 | μL |
| Total | 50 | mL |

### Cryopreservation Solution (Ratio: 3:1:1)

| | |
|---|---|
| DMEM/F-12 (gibco 11330032) | 3 mL |
| FBS | 1 mL |
| DMSO (sigma D4540) | 1 mL |
| Total | 50 mL |

### Cell Passage (First to Third)

When the cells were observed under microscope to converge to about 80%, the cells were transferred to a super-clean bench. A culture medium in a culture dish was discarded and the cells were washed twice with D-PBS. 1 mL of trypsin was added (closing over the bottom) and the cells were digested for about 3 min at 37° C. in a 5% CO2 incubator. When the cells were observed to be round under microscope, the digestion could be stopped. Then, 2 mL of a terminating culture medium was added, and the bottom was blown. The cell fluid was transferred into a 15 mL centrifuge tube and centrifuged at 1300r/min for 5 min. After centrifugation, a supernatant was discarded, and a myoblast culture medium was added into a sediment according to the passage ratio. After blowing and mixing well, the solution was evenly distributed to a new culture dish or culture bottle and cultured at 37° C. in a 5% CO2 incubator, with the liquid changed every other day.

### Cell Cryopreservation (First to Third)

When the cells were observed under microscope to converge to about 80%, the cells were transferred to a super-clean bench. A culture medium in a culture dish was discarded and the cells were washed twice with D-PBS. 1 mL of trypsin was added (closing over the bottom) and the cells were digested for about 3 min at 37° C. in a 5% CO2 incubator. When the cells were observed to be round under microscope, the digestion could be stopped. Then, 2 mL of a terminating culture medium was added, and the bottom was blown. The cell fluid was transferred into a 15 mL centrifuge tube and centrifuged at 1300r/min for 5 min. After centrifugation, a supernatant was discarded, and a DMEM/F-12 culture medium with the amount being a passage ratio multiplied by 500 μL was added into a sediment. After blowing and mixing well, 500 μL of a suspension and then 500 μL of a cryopreservation solution were added into each cryopreservation tube. Each cryopreservation tube was sealed with a sealing membrane and put in a cryopreservation box. The cryopreservation box was put in a refrigerator at −80° C. for 24 h and then put in a liquid nitrogen tank for long-term storage.

### 4. Distribution of Lnc4047 Cells

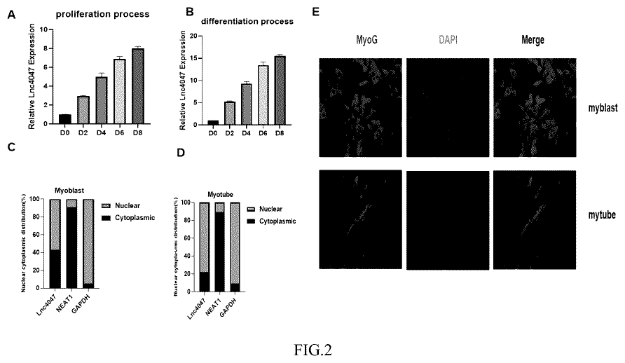

The purpose is for testing the expression characteristics of Lnc4047 in myoblast growth and differentiation stages. It was found by means of qRT-PCR test that these results suggested the possibility of Lnc4047 in participating in the proliferation and early differentiation of myoblasts of bos grunniens (A-B in FIG. 2). In order to analyze the distribution of Lnc4047 in nuclei and cytoplasms, myoblasts in a proliferation stage and myotubes in a differentiation stage were collected respectively. After nucleus-cytoplasm RNA separation, the mRNA expression levels of Lnc4047 in myoblasts and myotubes were measured, with NEAT1 and GAPDH as control genes specifically expressed in nuclei and cytoplasms, respectively. The qRT-PCR result of C in FIG. 2 showed that Lnc4047 was distributed in both nuclei and cytoplasms in a cell proliferation stage. However, with the process of myoblast differentiation, the content of Lnc4047 in cytoplasm gradually increased, from about 57% in myoblasts to 78% in myotubes (C-D in FIG. 2). The FISH technique was used to test the subcellular localization of Lnc4047, which was consistent with the result of Real-Time PCR. Lnc4047 was distributed in both nuclei and cytoplasms of undifferentiated cells, but was dominantly distributed in cytoplasms of differentiated cells (E in FIG. 2).

### 5. Lnc4047 Inhibited Myoblast Proliferation of Bos Grunniens

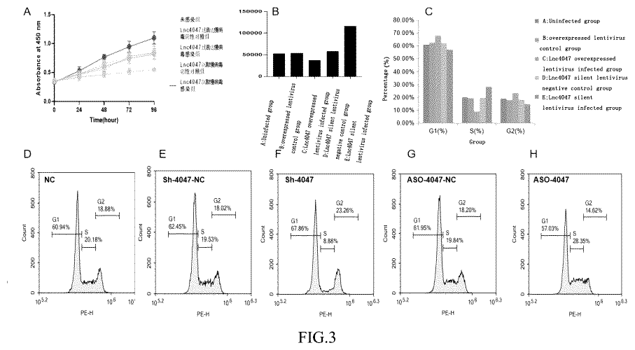

The effect of Lnc4047 on myoblast proliferation was tested using CCK-8. The results showed that compared with a control group, an overexpressed Lnc4047 could inhibit proliferation of myoblasts, while a silent Lnc407 could increase the proliferation ability of myoblasts (A in FIG. 3). The results of Edu cell proliferation experiments showed that compared with the control group, the proliferation of cells decreased significantly after overexpression of Lnc4047, while the proliferation of cells increased significantly after silencing of Lnc4047, indicating that Lnc4047 could inhibit cell proliferation (B in FIG. 3).

Cell cycle is an important index to characterize cell proliferation. Different stages (G0, G1, S, G2) of cell development due to the difference in DNA content can be distinguished by using a flow cytometer after staining with fluorescent dye propidine iodide (PI). The changes in cell cycle after overexpression/silencing of Lnc4047 were tested with the flow cytometer. The results showed that compared with the control group, overexpression of Lnc4047 significantly reduced the number of cells in phase S, while silencing of Lnc4047 significantly increased the number of cells in phase S (C-H in FIG. 3).

### 6. Lnc4047 Promoted Myoblast Differentiation of Bos Grunniens

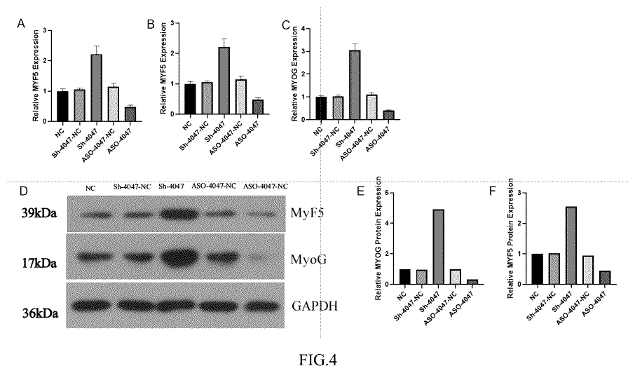

Myoblasts were infected with Lnc4047 overexpressed lentivirus or silenced lentivirus, and the mRNA expression levels of Lnc4047, MyoG and MyF5 were measured using Real-time PCR 48 h after infection. The results showed that compared with the control group, the relative expressions of Lnc4047 in groups C and E were obviously up/down-regulated after infection, indicating that the infection efficiency was high. Compared with the control group, the mRNA expression levels of differentiation marker genes MyoG and MyF5 in myoblasts were significantly increased after overexpression of Lnc4047, while the mRNA expression levels of MyoG and MyF5 in myoblasts were significantly decreased after silencing of Lnc4047. The protein expression levels of Lnc4047, MyoG and MyF5 were measured using Western blot 48 h after infection. The results showed that compared with the control group, the protein expression levels of MyoG and MyF5 in myoblasts increased significantly after overexpression of Lnc4047, but the protein expression levels of MyoG and MyF5 in myoblasts decreased significantly after silencing of Lnc4047 (FIG. 4).

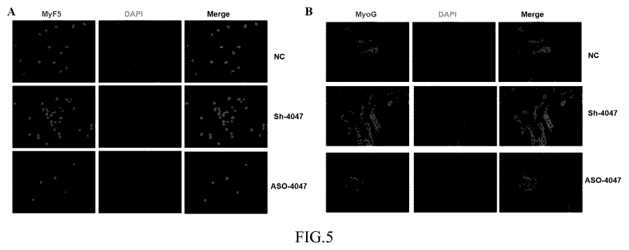

The changes in protein levels of MyoG and MyF5 after overexpression/silencing of Lnc4047 were measured using immunofluorescence. Compared with the control group, the protein expression levels of MyoG and MyF5 in myoblasts significantly increased after overexpression of Lnc4047, while the protein expression levels of MyoG and MyF5 in myoblasts significantly decreased after silencing of Lnc4047 (FIG. 5).

Although the above-mentioned examples provide a detailed description of the present invention, they are merely a part of, rather than all the examples of the present invention. Other examples may be obtained according to the examples without involving any inventive effort, and all the examples fall within the scope of protection of the present invention.

1. A bos grunniensLnc4047, wherein the nucleotide sequence of the bos grunniensLnc4047 is shown in SEQ ID No. 1.

2. An application of the bos grunniens Lnc4047 according to claim 1 in regulating myoblast proliferation of bos grunniens.

3. The application according to claim 2, wherein an overexpressed bos grunniens Lnc4047 inhibits myoblast proliferation of bos grunniens.

4. The application according to claim 2, wherein a silent bos grunniens Lnc4047 promotes myoblast proliferation of bos grunniens.

5. An application of the bos grunniens Lnc4047 according to claim 1 in regulating myoblast differentiation of bos grunniens.

6. The application according to claim 5, wherein an overexpressed bos grunniens Lnc4047 promotes myoblast differentiation of bos grunniens.

7. The application according to claim 5, wherein a silent bos grunniens Lnc4047 promotes myoblast proliferation of bos grunniens.
